(19) United States
(12) Patent Application Publication
(10) Pub. No.: US 2012/0277540 A1
(43) Pub. Date: Nov. 1, 2012

(54) Triangulation Concept for Minimally Invasive Access Surgery

(75) Inventor: Mark Russo, Plantsville, CT (US)

(73) Assignee: Tyco Healthcare Group LP, Mansfield, MA (US)

(21) Appl. No.: 13/442,006

(22) Filed: Apr. 9, 2012

Related U.S. Application Data
(60) Provisional application No. 61/480,073, filed on Apr. 28, 2011.

(51) Int. Cl. A61B 1/32

(52) U.S. Cl. 600/210

(57) ABSTRACT

A surgical system is adapted and configured for use in a minimally invasive surgical procedure. The surgical system includes surgical instruments that are configured and adapted to be inserted into an underlying body cavity in a first configuration and to transition to a second configuration within the body cavity to provide improved manipulation and visualization of internal body structures. A method of using the surgical system is also disclosed.

### CROSS REFERENCE TO RELATED APPLICATIONS

The present application claims the benefit of and priority to U.S. Provisional Application Ser. No. 61/480,073, filed on Apr. 28, 2011, the entire contents of which are incorporated herein by reference.

### BACKGROUND

1. Technical Field

The present disclosure relates generally to a surgical device for use in a minimally invasive surgical procedure. More particularly, the present disclosure relates to instrumentation for use in a minimally invasive surgical procedure that facilitate both the placement and use of the instruments within the surgical site.

2. Background of Related Art

A minimally invasive surgical procedure is one in which a surgeon enters a patient's body through one or more small openings in the patient's skin or a naturally occurring opening (e.g., mouth, anus, or vagina). As compared with traditional open surgeries, minimally invasive surgical procedures have several advantages and disadvantages. Minimally invasive surgeries include arthroscopic, endoscopic, laparoscopic, and thoracic surgeries. Advantages of minimally invasive surgical procedures over traditional open surgeries include reduced trauma and recovery time for patients.

However, some disadvantages include a lack of direct visualization of the surgical site and reduced dexterity of instruments, as compared to traditional open surgeries. In particular, the simultaneous manipulation of the viewing instrument and surgical instruments that are inserted into the opening may be complicated. One complication arises from the difficulty in visualizing surgical instruments on a monitor that is operably coupled to the viewing instrument.

One surgical technique used to increase the ability of the surgeon to visualize and access critical anatomy is triangulation. Triangulation is a principle in which the surgical instrument and the viewing instrument are held so that their tips form the apex of an imaginary triangle. In particular, the viewing instruments may be in the middle of the surgical field, and the surgical instruments may be angled with respect to the viewing instrument as to form an imaginary triangle.

In minimally invasive surgical procedures through a single opening, straight and rigid surgical instruments are inserted through a single incision. To control the instruments, a surgeon often crosses his hands. The lack of triangulation makes visualization and access of critical anatomy potentially difficult. Furthermore, it is desirable to coordinate the positions of end effectors of the surgical instruments.

Consequently, a continuing need exists for improved minimally invasive surgical devices.

### SUMMARY

Disclosed herein is a surgical system for use during a minimally invasive surgical procedure. In particular, a minimally invasive surgical triangulation system is disclosed. The minimally invasive surgical triangulation system includes a seal anchor member that is transitionable between a first condition and a second condition.

The seal anchor member includes a leading portion, a trailing portion, and an intermediate section positioned between the leading and trailing portions. At least two ports extend longitudinally through the intermediate section. Each port defines a longitudinal axis through which a surgical instrument is placed.

The surgical instrument includes a shaft that is rotatable about the longitudinal axis of the port. By rotating the shaft of the surgical instrument within the port, the orientation of the surgical instrument is transitioned between first and second orientations. When the surgical instruments have the first orientation, the triangulation system is in a first condition. Conversely, the triangulation system is in the second condition when the surgical instruments are in the second orientation.

Moreover, the minimally invasive surgical triangulation system defines a width that corresponds to the orientation of the shaft of the surgical instrument within the port. In an embodiment, the shaft of the surgical instrument defines a contour that is generally S-shaped. Depending on the orientation of the shafts of the surgical instruments within the ports, the shafts of the surgical instruments collectively define either a bulb shape or a coiled, helical shape. When the shafts define a bulb shape, the greatest distance between any two points along the surface of the shafts is greater than when the shafts define a coiled or helical shape. Consequently, the width of the triangulation system is greater in the second condition in which the shafts define a bulb shape than when the triangulation system is in the first condition and the shafts define a coiled or helical shape.

Placement of the triangulation system within a body opening defined within tissue is facilitated when the triangulation system is in the first condition as compared to when it is in the second condition. Once placed inside the body opening, the surgical instruments are triangulated with respect to one another by rotating the shafts of the surgical instrument and transitioning the shafts to define the bulb shape. Once the desired surgical procedure is performed, the triangulation system is transitioned back to the first condition to facilitate removal of the triangulation system from the body opening.

Methods of using the triangulation system are also disclosed including providing the triangulation system, placing the triangulation system while in the first condition having the reduced profile or width into a body opening to access an underlying body cavity. Once within the triangulation system is placed within body opening, the surgical instruments are triangulated with respect to one another by actuating the surgical instruments, thereby transitioning the triangulation system to the second condition. Once the triangulation system is the second condition, a desired surgical procedure is performed. Upon completion of the desired surgical procedure, the triangulation system is transitioned back to the first condition to facilitate the removal of the triangulation system from the surgical site.

These and other features of the current disclosure will be explained in greater detail in the following detailed description of the various embodiments.

### BRIEF DESCRIPTION OF THE DRAWINGS

Various embodiments of the present disclosure are described hereinbelow with reference to the drawings, wherein:

FIG. 1 is a front perspective view of a seal anchor member shown positioned relative to tissue;

FIG. 2 is a front perspective view of a triangulation system including the seal anchor member of FIG. 1 shown with surgical instruments placed therein in a first condition;

FIG. 3 is a front perspective view of the triangulation system of FIG. 2 shown in a second condition;

FIG. 4A is a cross-sectional view of the triangulation system of FIG. 2 taken along section line shown in the first condition;

FIG. 4B is a bottom view of the triangulation system of FIG. 2 shown in the first condition;

FIG. 4C is a schematic view illustrating movement of the surgical instruments;

FIG. 5A is a cross-sectional view of the triangulation system of FIG. 2 shown in a second condition; and

FIG. 5B is a bottom view of the triangulation system of FIG. 2 shown in the second condition.

### DETAILED DESCRIPTION OF THE EMBODIMENTS

Particular embodiments of the present disclosure will be described herein with reference to the accompanying drawings. As shown in the drawings and as described throughout the following descriptions, and as is traditional when referring to relative positioning on an object, the term “proximal” will refer to the end of the apparatus that is closest to the clinician during use, and the term “distal” will refer to the end that is farthest from the clinician during use.

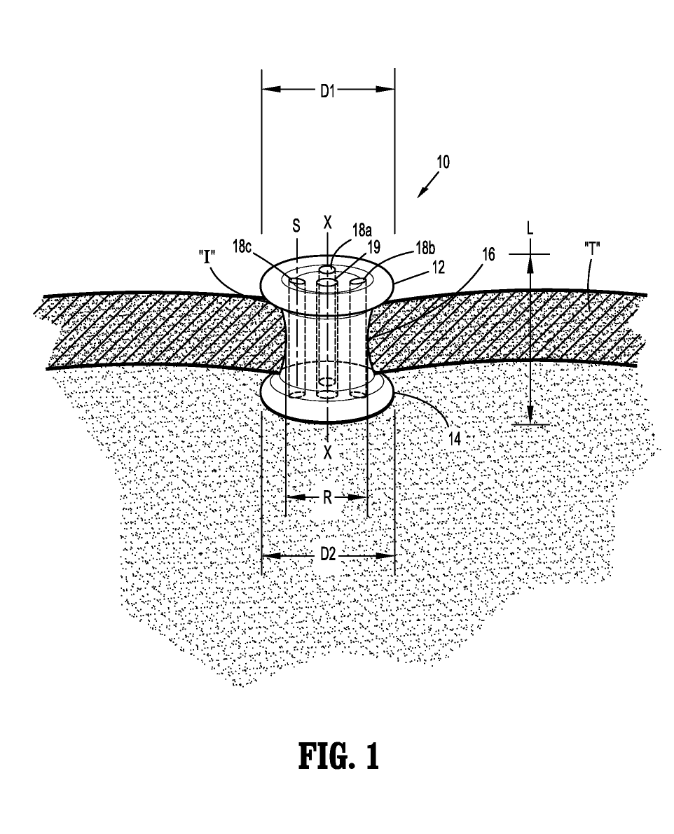

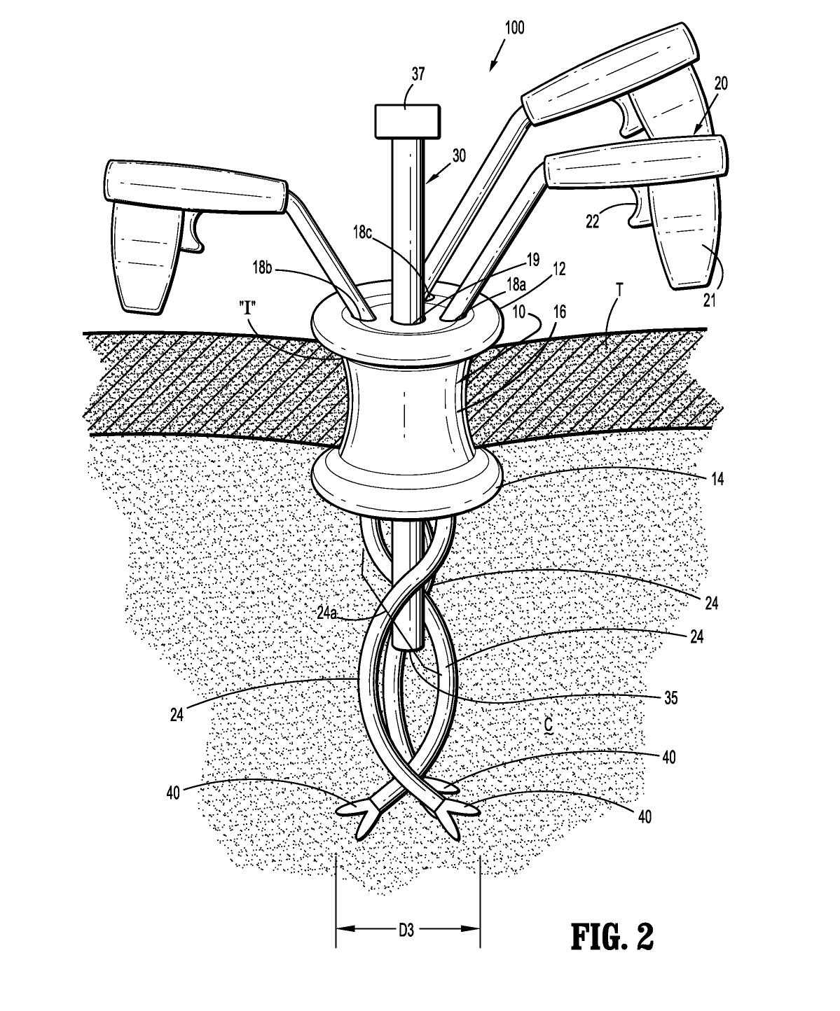

With reference to FIG. 1, a seal anchor member 10 for use in a minimally invasive surgical procedure will now be described. The seal anchor member 10 defines a longitudinal axis X and has respective trailing (or proximal) and leading (or distal) ends 12, 14, and an intermediate portion 16 disposed between the trailing and leading ends 12, 14. Seal anchor member 10 includes one or more ports 18a-c, which extend longitudinally between trailing and leading ends 12, 14, respectively. Each of the ports 18a-c defines a longitudinal axis S. A centrally disposed lumen 19 may be positioned among the one or more ports 18a-c. The lumen 19 longitudinally extends through seal anchor member 10 and is configured and adapted to receive a viewing instrument 30 (FIG. 2) therethrough.

The trailing end 12 of the seal anchor member 10 defines a first diameter D1, and the leading end 14 of the seal anchor member 10 defines a second diameter D2. The intermediate portion between the trailing and leading ends 12, 14 defines a radial dimension R. The radial dimension R may be less than the first and second diameters D1, D2 of the trailing and leading ends 12, 14, respectively, and may vary along length L of the seal anchor member 10 to define a substantially hour-glass configuration.

The seal anchor member 10 is insertable within tissue tract I defined within body opening I of tissue T. The hour-glass configuration of the intermediate section 16 of the seal anchor member 10 may facilitate anchoring of the seal anchor member 10 within the body opening I. Furthermore, the seal anchor member 10 is configured and adapted to establish a sealing relation with the tissue T. An example of such a seal anchor member 10 is illustrated in U.S. Pat. Pub. 2009/0093752, the entire contents of which are hereby incorporated by reference.

A triangulation system 100 will now be described with respect to FIGS. 2-5B. The triangulation system 100 includes the seal anchor 10 and at least two surgical instruments 20 placed within the one or more ports 18a-c of the seal anchor member 10. A viewing instrument 30 may be placed within the centrally disposed lumen 19 and centered between the surgical instruments 20 that are placed within ports 18. The viewing instrument 30 includes a viewing portion 35, e.g., lens, and a monitor or eyepiece 37 for remotely viewing the surgical site within body cavity C.

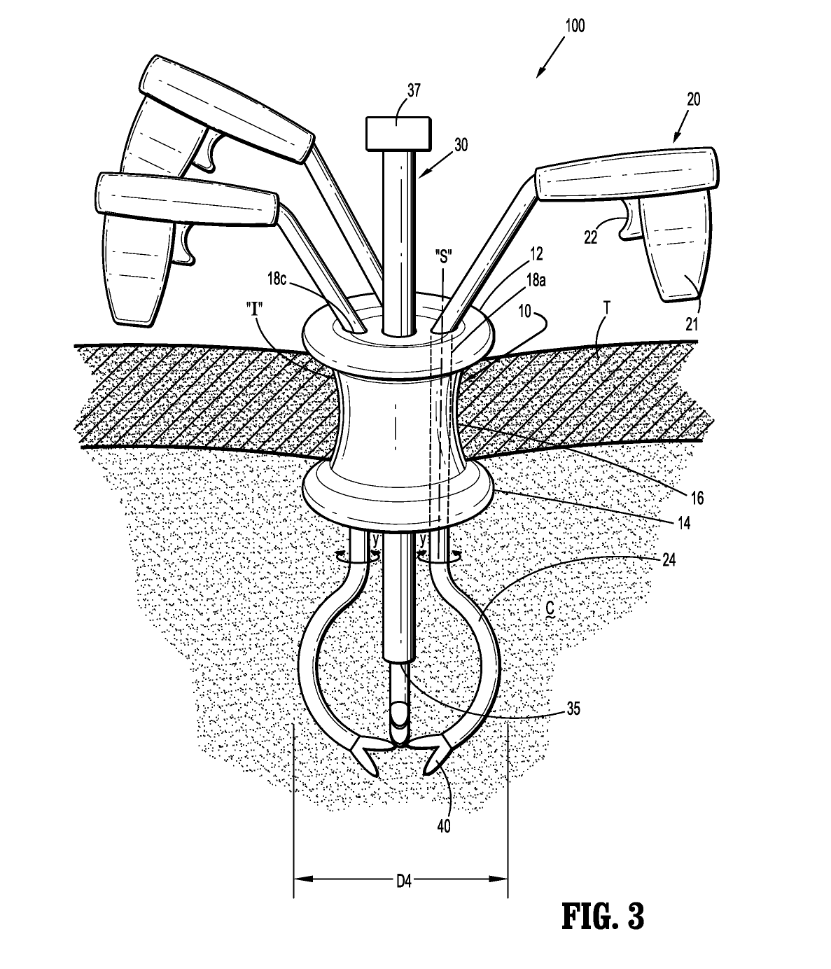

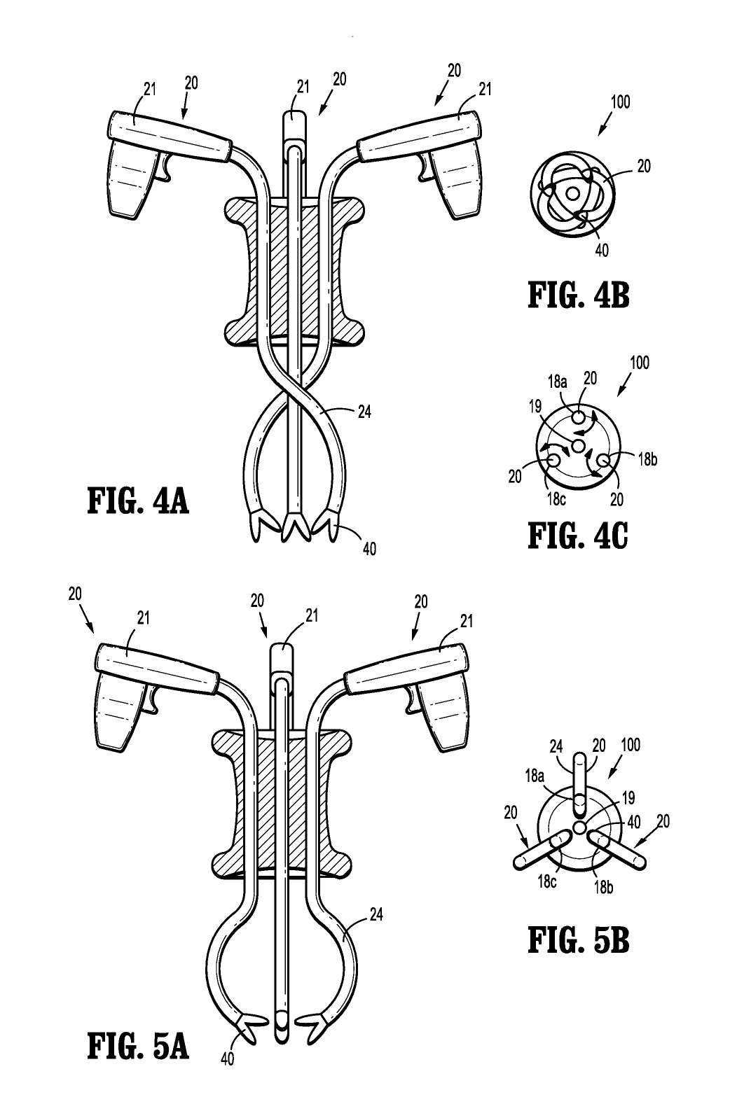

The triangulation system 100 is configured and adapted to transition between a first condition as shown in FIGS. 2, 4A-B and a second condition as shown in FIGS. 3, 5A-5B. Each surgical instrument 20 of the triangulation system 100 includes a specially shaped shaft 24 to facilitate the transition between the first and second conditions. As shown best in FIGS. 4A and 5A, the shafts 24 of the surgical instrument 20 are generally S-shaped. As will be described below, the S-shaped configuration of the shafts 24 facilitate transitioning of the triangulation system 100 between the first condition and the second condition. In the first condition, the triangulation system 100 has a relatively narrower profile as compared to when the triangulation system 100 is in the second condition.

The triangulation system 100 may be placed within the body opening I with the surgical instruments 20 placed within the ports 18a-c such that the triangulation system 100 is in the first condition. Alternatively, the surgical instruments 20 may be inserted into the ports 18a-c subsequent to the placement of the seal anchor member 10 into the body opening I. In particular, once the seal anchor member 10 is placed within the body opening I, the surgical instruments 20 may be placed within the ports 18a-c such that the triangulation system 100 is in the first condition. Removal of the triangulation system 100 may be accomplished by removing the entire triangulation system 100 with the surgical instruments 20 placed within the ports 18a-c such that the triangulation system 100 is in the first condition. Alternatively, the surgical instruments 20 may be removed from the ports 18a-c prior to the removal of the seal anchor member 10.

In the first condition, the shafts 24 of the surgical instruments 20 collectively form a generally helical or coiled configuration with each of the shafts 24 in a first orientation with respect to the longitudinal axis S of the ports 18a-c in which the shafts 24 are positioned. As shown best in FIG. 2, when the triangulation system 100 is in the first condition, the shafts 24 of the surgical instruments 20 collectively define a generally helical or coiled configuration, which minimizes the profile or width of the triangulation system 100. In the first condition, the distance D3, defining the greatest distance between any two points on the surfaces of the shafts 24 is minimized to facilitate placement of the triangulation system within body opening I of tissue T. In particular, an elongated generally straight section 24a of the shaft 24 is inwardly oriented such that the shaft 24 does not extend past the leading end 14 of the seal anchor member 10.

As shown in FIGS. 2, 3, 4A, and 5A, each of the surgical instruments 20 includes an actuation mechanism 21 including a handle 21 and an actuator or trigger 22. Actuation of the actuation mechanism 21 by depressing the trigger 22 causes actuation of the end effectors 40 of the surgical instruments 20. The surgical instruments are rotatable within ports 18a-c of the seal anchor member 10. In particular, a surgeon may rotate shafts 24 of the surgical instruments 20 within the ports 18a-c along bi-directional arrow Y (FIGS. 3 and 4C). Due to the shape of the shafts 24, depending on the orientation of the shafts 24, the rotation of the shafts 24 in a first direction will cause either an increase or a decrease in the distance between the farthest two points on the surfaces of any two of the shafts 24.

Transition of the triangulation system 100 to the second condition, as shown in FIGS. 3 and 4C, is accomplished by rotating surgical instruments 20 in the direction of arrow Y about longitudinal axis S of the ports 18 in which the surgical instruments 20 are placed. In the second condition, the shafts 24 of the surgical instruments collectively define a bulb or bulbous shape. In particular, the greatest distance between the farthest two points on the surfaces of the shafts 24 defines a distance D4 that is greater than distance D3. The generally S-shaped contour of the shaft 24, which includes an elongated generally straight section 24 facilitates the transition between the first and second conditions of the triangulation system. In an embodiment, as shown in FIG. 3, by rotating the shaft 24 of the surgical instrument 20, the shaft 24 is outwardly oriented such that a portion of the shaft 24 extends past the leading end 14 of the seal anchor member 10.

As shown in FIGS. 3, 5A, and 5B when the triangulation system is in the second condition, the end effectors 40 of the surgical instruments 20 are triangulated with respect to one another. As shown in FIG. 3, by triangulating the end effectors 40 with respect to each other, visualization of the positioning of the end effectors 40 is facilitated by bringing the end effectors within the field of view of the viewing portion 35 of the viewing instrument 30.

A method of using the triangulation system 100 will now be described. The triangulation system 100 is provided and is placed in the body opening I of tissue T to access the body cavity C. It is advantageous that the profile or width of the triangulation system 100 be as small as possible such that the triangulation system 100 can be placed within a relatively small body opening I without necessitating increasing the size of the body opening I. To this end, the triangulation system 100 is inserted into the body opening I while in the first condition in which the shafts 24 of the surgical instruments 20 collectively define a generally coiled or helical configuration (FIG. 2).

Once placed, shafts 24 of the surgical instrument 20 are placed in the body cavity C, the surgical instrument 20 can be actuated to cause triangulation of the end effectors 40 with respect to one another and the viewing portion 35 of viewing instrument 30. By actuating the surgical instruments 20, the shafts 24 of the surgical instruments 20 are rotated along bi-directional arrow Y as shown in FIGS. 3 and 4C, thereby transitioning the triangulation system 100 to the second condition in which the shafts 24 of the surgical instrument 20 collectively define an uncoiled, bulbous shape. In the second condition, visualization of the surgical site is facilitated since the end effectors 40 of the surgical instruments are triangulated with respect to one another and the viewing portion 35 of the viewing instrument 30.

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to those precise embodiments, and that various other changes and modifications may be effected therein by one skilled in the art without departing from the scope or spirit of the disclosure.

What is claimed is:

1. A minimally invasive surgical triangulation system, comprising:
a seal anchor member including:
a leading portion;
a trailing portion;
an intermediate section positioned between the leading and trailing portions; and
at least two ports, each port longitudinally extending through the intermediate section and the leading and trailing portions, each port defining a longitudinal axis; and
at least two surgical instruments, each surgical instrument configured and adapted to be placed within one of the ports, the surgical instrument including a shaft, the shaft rotatable about the longitudinal axis of the port to transition between a first and a second orientation, the minimally invasive surgical triangulation system defining a first condition corresponding to the first orientation and the defining a second condition corresponding to the second orientation.

2. The minimally invasive surgical triangulation system of claim 1, wherein the minimally invasive surgical triangulation system defines a width, the width corresponding to the orientation of the shaft of the surgical instrument within the port.

3. The minimally invasive surgical triangulation system of claim 2, the shaft of the surgical instrument defines a contour such that the width of the minimally invasive triangulation system is wider in the second condition than when in the first condition.

4. The minimally invasive surgical triangulation system of claim 3, wherein the contour of the shaft of the surgical instrument is generally S-shaped.

5. The minimally invasive surgical triangulation system of claim 1, wherein the surgical instrument includes an actuator to facilitate transitioning of the shaft of the surgical instrument between the first and second orientations.

6. The minimally invasive surgical triangulation system of claim 1, wherein insertion of the seal anchor within a body opening within a tissue is facilitated in the first position.

7. The minimally invasive surgical triangulation system of claim 1, wherein the shaft of the surgical instrument includes an end effector coupled to a distal end of the shaft.

8. The minimally invasive surgical triangulation system of claim 7, wherein the end effectors of the surgical instruments are triangulated with respect to one another when the minimally invasive surgical triangulation system is in the second condition.

9. A method of performing a minimally invasive surgery, comprising:
providing a triangulation system including:
a seal anchor member including:
a leading portion;
a trailing portion;
an intermediate section positioned between the leading and trailing portions; and
at least two ports, each port longitudinally extending through the intermediate section and the leading and trailing portions, each port defining a longitudinal axis; and
at least two surgical instruments, each surgical instrument configured and adapted to be placed within one of the ports, the surgical instrument including a shaft, the shaft rotatable about the longitudinal axis of the port to transition between a first and a second orientation, the minimally invasive surgical triangulation system defining a first condition corresponding to the first orientation and the defining a second condition corresponding to the second orientation;
placing the triangulation system while in the first condition into a body opening within a tissue to access an underlying body cavity;
transitioning the triangulation system into the second condition;
performing a desired surgical procedure;
transitioning the triangulation system back to the first condition; and
removing the triangulation system from the body opening.
